(19) United States
(12) Patent Application Publication
(10) Pub. No.: US 2019/0343307 A1
(43) Pub. Date: Nov. 14, 2019

(54) TRAVEL PILLOW

(71) Applicant: Angela Rose Cullins, Vancouver (CA)

(72) Inventor: Angela Rose Cullins, Vancouver (CA)

(21) Appl. No.: 15/976,792

(22) Filed: May 10, 2018

(51) Int. Cl. A47G 9/10; A61M 21/02

(52) U.S. Cl. CPC A47G 9/1045; A61M 2021/0016; A61M 21/02; A47G 9/1036

(57) ABSTRACT

The present invention relates generally to travel pillows and, in particular, to a travel pillow with integrated comfort and therapeutic features.

### FIELD OF THE INVENTION

The present specification relates generally to travel pillows and, in particular, to a travel pillow with integrated comfort and therapeutic features.

### BACKGROUND OF THE INVENTION

When traveling, particularly during long stretches in a car or on an airplane, it is common for people to experience neck pain from holding their head in an upright position for an extended period, or from improper positioning or insufficient adjustment of a headrest. Travel pillows which rest upon the back of the neck and wrap around the shoulders to engage the neck and support the head are thus used to try and alleviate this discomfort.

However, rather than simple adjustment and support for the head and neck, it would be advantageous for a travel pillow to provide additional comfort and/or therapeutic features. Ideally, such features would be incorporated into the pillow in such a way as to permit user modification.

While there are some travel pillows known in the art, it would be desirable to have a travel pillow which mitigates some of the known disadvantages or provides some greater advantages.

Accordingly, there remains a need for improvements in the art.

### SUMMARY OF THE INVENTION

In accordance with an aspect of the invention, there is provided a travel pillow with integrated comfort and therapeutic features.

According to an embodiment of the invention, there is provided a travel pillow, comprising: a pair of side cushions, each side cushion formed into a shape with a concavity at a bottom side for engagement with a user's shoulders, and each side cushion containing one or more resealable enclosures on one or more sides; a detachable neck/lumbar support engaged to a rear side of each of the side cushions, the support positioned to be disposed against the user's neck when the side cushions are engaged with the user's head and when the travel pillow is resting on a seat back; a connecting strap, the connecting strap engaged with each of the side cushions at a location different from the detachable support; and one or more therapeutic elements disposed within at least one of the resealable enclosures.

According to a further embodiment of the invention, the neck/lumbar support may further include one or more therapeutic components, the therapeutic components including one or more of: a heating element, a cooling element, a magnet, a massager.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1A is a front view of a pair of cushions for a travel pillow according to an embodiment;

FIG. 1B is a front view of a detachable lumbar/neck support for a travel pillow according to an embodiment;

FIG. 2 is a front view of a travel pillow formed from combining the cushions of FIG. 1A and the detachable support of FIG. 1B;

FIG. 3 is a rear view of the travel pillow of FIG. 2; and

FIG. 4 is a right side view of the travel pillow of FIG. 2.

Like reference numerals indicated like or corresponding elements in the drawings.

### DETAILED DESCRIPTION OF THE EMBODIMENTS

The present invention relates generally to travel pillows and, in particular, to a travel pillow with integrated comfort and therapeutic features.

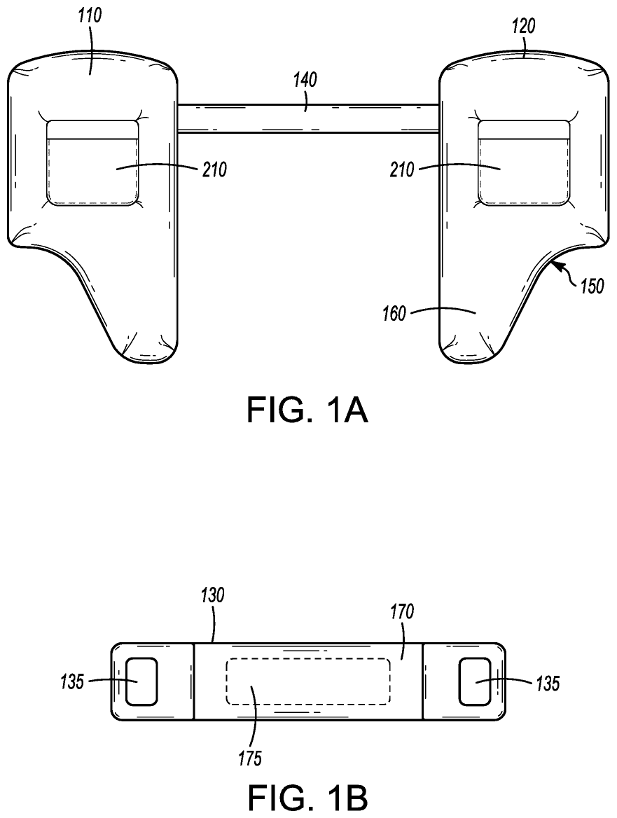

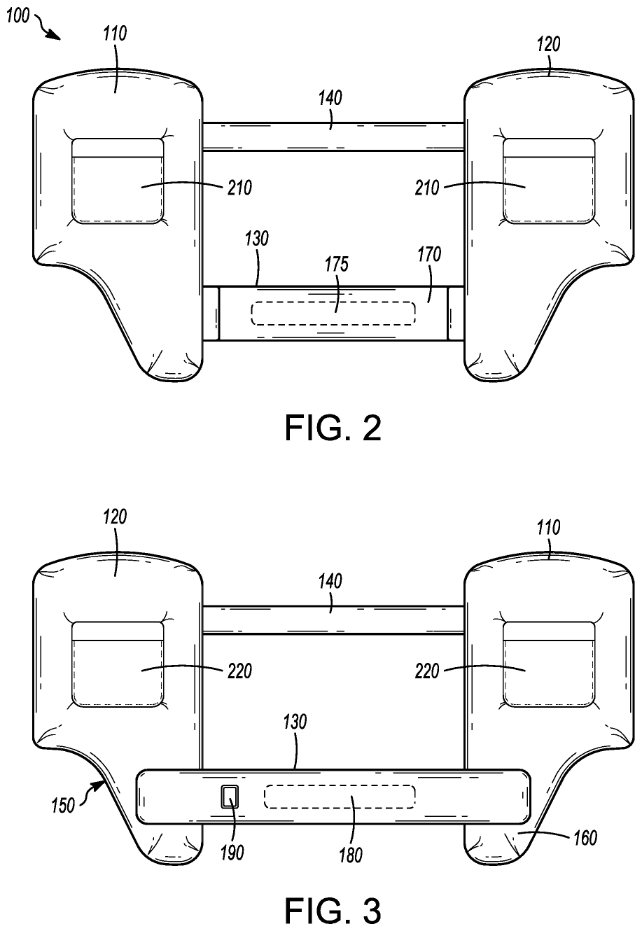

According to an embodiment as shown in FIG. 2, a travel pillow 100 is formed from two side cushions 110, 120 connected by a connecting strap 140 as shown in FIG. 1A. A removable neck/lumbar support 130 as shown in FIG. 1B is removably secured to each side cushion 110 and 120 by a fastener 135 (hook-and-loop, snaps, magnets, etc.) to provide neck support when the travel pillow 100 is used with a headrest or seat back. Support 130 and/or strap 140 may further be used to form a carrying case or handle for travel pillow 100.

Strap 140 may be elasticized to assist in holding cushions 110, 120 together for carrying or storage, as well as to assist in holding travel pillow 100 in position on the head while in use. Strap 140 may be made from a thin band of fabric or similar material in order to minimize size and impact on the user while the travel pillow 100 is in use.

Each side cushion 110, 120 may be formed from cotton wadding, medium- or high-density foam, memory foam, down, down substitute, or a similar material which provides support to the user's head while remaining moderately deformable. This material is then enclosed within a fabric cover, made of cotton or a similar fabric to form the cushion. Each side cushion 110, 120 may additionally by covered by a separate removable cover to permit cleaning. Preferably, all materials used may be hypoallergenic.

The side cushions 110, 120 each have a generally flat or slightly rounded interior side that rests against the user's head while in use, and a concavity 150 across the exterior side and rear side to provide space for the user's shoulders. Further, side cushions 110, 120 may include an extension 160 at the bottom of the front and/or rear side to engage the front and/or rear of the user's shoulders and neck. The overall shape of cushions 110, 120 as defined by concavity 150 and extension 160 may be such that proper ergonomic support is provided to the user e.g. the weight of the cushions is distributed to the user's shoulders, as opposed to the head/neck area.

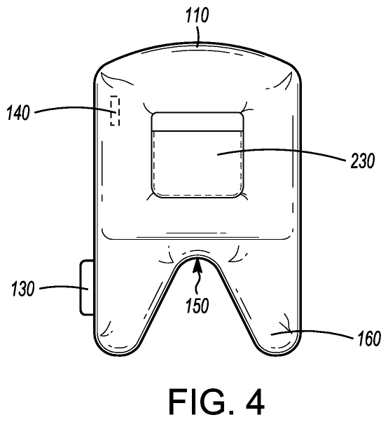

Taking side cushion 110 as an example as shown in FIGS. 2, 3 and 4, the exterior of side cushion 110 may further include one or more resealable enclosures 210, 220 and 230, located on any or all of the interior side, front side, exterior side, or back side of side cushion 110. Sealable enclosures 210, 220, 230 may be used for insertion of magnets, heating pads, cooling pads, essential oil sachets, or other therapeutic or homeopathic substances by the user. The enclosures 210, 220, 230 may by sealed by hook-and-loop fasteners, snaps, buttons, magnets or other methods as known in the art. However, where the enclosures 210, 220, 230 are meant for use with magnets, a magnetic enclosure seal may be less desirable.

Enclosures 210, 220, 230 may be external pockets secured to the fabric covering of side cushion 110 or may be integrated pockets built-in to the fabric covering. The latter may be preferable for enclosures on the interior side of side cushion 110 to minimize the impact of the contents of the enclosure 210 on the user's head when the travel pillow 100 is in use. Where a removable cover is used with the side cushions as described above, the enclosures may instead be formed as part of the removable cover, preferably on the interior surface of the cover facing the side cushions.

The enclosures 210, 220, 230 may be positioned in different locations on the cushion to provide different therapeutic options. For example, an enclosure may be placed at a location towards the top of the cushion to provide therapeutic options for the ears or eyes. Alternatively, an enclosure may be located toward the bottom of the cushion to provide therapeutic options for the shoulder, collarbone and neck regions. Multiple enclosures may also be provided in multiple positions for a greater variety of therapeutic options.

Removable support 130, as best shown in FIGS. 1B, 2 and 3 may be formed from a section of flexible fabric with a neck/lumbar cushion 170 formed of a deformable support material similar to side cushions 110 and 120. The cushion 170 may be dimensioned roughly to the width of an adult neck, or to a child neck, in a smaller version for use by children or smaller adults. Removable support 130 may also include a therapeutic component 175, as well as a USB port 190 or similar battery charger to provide power directly to the therapeutic component 175 or a separate battery 180. Therapeutic component 175 may also include a heating element, a cooling element, a massager or vibrator, magnets or a combination thereof disposed within or on the surface of cushion 170. Additionally, therapeutic component 175 may be enclosed in a removable pocket, similar to pockets 210, 220, 230 on the cushions 110, 120, allowing for therapeutic component 175 to be removed or replaced.

In operation, removable support 130 may remain attached to side cushions 110 and 120 for use solely as a head/neck support pillow when traveling (i.e. airline seat bus seat) or other occasions when a support pillow may be useful (i.e. office chair, armchair, sofa, etc.). If the user desires, removable support may be detached, permitting the use of the travel pillow 100 alone for head/neck support, either due to personal desire or comfort. When detached, removable support 130 may be used in a lumbar support position at the user's lower back, with or without activation of the therapeutic component 175, and with or without the travel pillow 100.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A travel pillow, comprising:
a pair of side cushions, each side cushion formed into a shape with a concavity at a bottom side for engagement with a user's shoulders, and each side cushion containing one or more resealable enclosures on one or more sides;
a detachable neck/lumbar support engaged to a rear side of each of the side cushions, the support positioned to be disposed against the user's neck when the side cushions are engaged with the user's head and when the travel pillow is resting on a seat back;
a connecting strap, the connecting strap engaged with each of the side cushions at a location different from the detachable support; and
one or more therapeutic elements disposed within at least one of the resealable enclosures.

2. The travel pillow of claim 1, wherein the neck/lumbar support further includes one or more therapeutic components, the therapeutic components including one or more of: a heating element, a cooling element, a magnet, a massager.

3. The travel pillow of claim 1, wherein the therapeutic elements are selected from one or more of: magnets, heating pads, cooling pads and essential oil sachets.

4. The travel pillow of claim 1, further including a removable cover for each of the side cushions.

5. The travel pillow of claim 1, wherein the flexible panel and detachable strap form a carry case for the travel pillow.

6. The travel pillow of claim 1, wherein the resealable enclosures are attached externally to the side cushions.

7. The travel pillow of claim 1, wherein the resealable enclosures are formed internally to the side cushions.

8. The travel pillow of claim 2, wherein the neck/lumbar support further includes a rechargeable power supply for the one or more therapeutic components.

9. The travel pillow of claim 2, wherein the one or more therapeutic components is removable from the neck/lumbar support.

10. A travel pillow, comprising:
a pair of side cushions, each side cushion formed into a shape with a concavity at a bottom side for engagement with a user's shoulders;
a detachable neck/lumbar support engaged to a rear side of each of the side cushions, the support positioned to be disposed against the user's neck when the side cushions are engaged with the user's head and when the travel pillow is resting on a seat back;
a connecting strap, the connecting strap engaged with each of the side cushions at a location different from the detachable support.

11. The travel pillow of claim 10, wherein each side cushion further comprises one or more resealable enclosures on one or more sides, with one or more therapeutic elements disposed within at least one of the resealable enclosures.

12. The travel pillow of claim 10, wherein the neck/lumbar support further includes one or more therapeutic components, the therapeutic components including one or more of: a heating element, a cooling element, a magnet, a massager.

13. The travel pillow of claim 11, wherein the therapeutic elements are selected from one or more of: magnets, heating pads, cooling pads and essential oil sachets.

14. The travel pillow of claim 10, further including a removable cover for each of the side cushions.

15. The travel pillow of claim 10, wherein the flexible panel and detachable strap form a carry case for the travel pillow.

16. The travel pillow of claim 14, wherein each removable cover further comprises one or more resealable enclosures, with one or more therapeutic elements disposed within at least one of the resealable enclosures.

17. The travel pillow of claim 16, wherein the resealable enclosures are formed on the surface of the removable cover facing the side cushions.

18. The travel pillow of claim 12, wherein the neck/lumbar support further includes a rechargeable power supply for the one or more therapeutic components.

19. The travel pillow of claim 12, wherein the one or more therapeutic components is removable from the neck/lumbar support.
